(19) United States
(12) Patent Application Publication
(10) Pub. No.: US 2009/0104214 A1
(43) Pub. Date: Apr. 23, 2009

(54) Synthetic peptides containing the motif "YWWLXP" as anthrax toxin antidotes

(76) Inventors: Paolo Neri, Siena (IT); Luisa Bracci, Siene (IT); Alessandro Pini, Via Banchi di Sotto (IT)

Correspondence Address: JUNEAU PARTNERS, P.O. BOX 2516, ALEXANDRIA, VA, 22301, US

(21) Appl. No.: 11/547,092

(22) PCT Filed: Mar. 25, 2005
(86) PCT No.: PCT/IT2005/000168

§ 371 Date: Sep. 21, 2007

(30) Foreign Application Priority Data
Mar. 30, 2004 (FI) 2004A000076

(51) Int. Cl. A61K 39/00; C07K 7/00; A61K 38/10; A61K 38/08

(52) U.S. Cl. 424/185.1; 530/328; 530/329; 530/327; 514/16; 514/14

(57) ABSTRACT

Specific inhibitors, in Multiple Antigen Peptide form, which inhibit the interaction between protective antigen and lethal factor and neutralize anthrax toxin in vivo. Anti protective antigen peptides were selected from a phage library by competitive panning with lethal factor. Selected 12mer peptides were synthesized in tetra-branched form and systematically modified to achieve peptides with higher affinity and inhibitory efficiency.

The dramatic increase in research for new anthrax therapeutics was prompted by potential use of Bacillus anthracis as a bio-weapon. A determinant goal is the discovery of new drugs to block the action of anthrax toxin, the dominant virulence factor of B. anthracis. Anthrax toxin consists of three proteins produced by the bacterium, protective antigen (PA), edema factor (EF) and lethal factor (LF), which when combined form two potent toxins: edema toxin and lethal toxin.

Protective antigen is an 83 kDa protein proteolytically activated by a furin-like protease on binding to a cellular receptor (Anthrax Toxin Receptor)¹. Proteolytic removal of the N-terminal 20 kDa fragment allows the remaining receptor-bound portion (PA63) to oligomerize into a heptameric ring. The heptamer binds LF and EF and translocates them into the cytosol, where they can exert their biological activity^{2,3}. EF is a calmodulin-dependent adenylate cyclase⁴. LF is a Zn-dependent metalloprotease that cleaves mitogen-activated protein kinase-kinases, leading to inhibition of several cell signalling pathways⁵ and producing major, often lethal damage to infected individuals. Lethal factor and the complex formed by PA and LF are therefore the main targets of therapeutic agents currently studied world-wide.

The main problem with anthrax therapy is the low efficiency of antibiotic treatment when it is not initiated in the first hours after exposure⁶. Once the bacteria have secreted large amounts of anthrax toxin, treatment with antibiotics is largely ineffective. No effective therapy against anthrax toxin is currently available. New synthetic or recombinant molecules to be used with antibiotics in combination therapy therefore have high priority. The most promising molecules that could lead to new anti-anthrax drugs are: i) anti-PA recombinant antibodies, derived from murine monoclonal antibodies⁷; ii) human monoclonal antibodies that neutralize toxin activity in vitro and in vivo⁸; iii) the soluble form of the extracellular von Willebrand factor type A domain of the anthrax toxin receptor, which inhibits anthrax toxin activity in a cell-based assay¹; iv) a mutant recombinant PA which oligomerizes with native PA giving rise to non functional oligomers⁹; v) small molecule (non peptide) inhibitors of LF enzyme activity¹⁰⁻¹¹, vi) the small stable furin inhibitor hexa-D-arginine amide which delays anthrax toxin-induced toxaemia¹², and vii) peptides derived from a phage library, that block anthrax toxin assembly when exposed in multiple copies on a polyacrylamide backbone¹³ (patent application N US2003108556).

Here, we selected anti-PA63 ligands from a large phage peptide library by competitive panning, using recombinant LF. This enabled single step recovery of phages carrying peptides that interfere with PA-LF binding. We identified several sequences that inhibit PA-LF binding and also inhibit their toxicity towards cultured cells. Two peptide sequences were synthesized in linear and a tetra-branched (Multiple, Antigen Peptide, MAP) form¹⁴, with four identical molecules linked to a lysine core. We previously demonstrated that MAP molecules are more effective as in vivo antidotes than linear peptides¹⁵ due to their general higher stability to blood proteases and peptidases¹⁶. The two lead MAP sequences were subjected to systematic modification by alanine scanning, progressive shortening and randomization of residues found less critical for binding. Several second generation MAPs with higher affinity and inhibiting potency were selected. Branched peptide inhibitors were tested in an animal model. Some of them completely inhibited anthrax toxin lethality.

### SUMMARY OF THE INVENTION

Now the Authors have found new peptides that are able to bind PA and, at the same time, are effective in blocking the interaction of PA with LF, i.e. the interaction of the two protein components which naturally form the lethal toxin of B. anthracis, with an IC50 that is compatible with the in vivo use of present peptides, which operate as effective therapeutic agents having the ability to neutralise the main cause of death due to B. anthracis infection.

Therefore, the present invention relates to the linear peptides comprising the amino acid sequence NH₂-YWWLX′P-COOH (Seq.Id.1), where X′ is an amino acid, and pharmaceutically acceptable salts thereof.

The invention further relates to the aforesaid peptides in Multiple Antigen Peptide (MAP) form, with formula (I) set out below, the use of said peptides, both in linear form and in MAP form, for the preparation of pharmaceutical compositions for treatment of the infection caused by B. anthracis, and the pharmaceutical compositions comprising such peptides.

Characteristics and advantages of the invention will be illustrated in detail in the description that follows.

### FIGURES

FIG. 1: Histograms showing the percentage of binding between LF and PA63 in the presence of LIN (linear) and MAP (tetra-branched) peptides used at three different concentrations, i.e. in molar ratio of 100, 10 and 1 relative to PA63, as described in Example 1. The 100% represents the binding of PA63 on LF in the absence of the peptides.

FIG. 2: Histograms showing percentage of cell viability as measured by the MTT assay such as described in example 1. Control: Untreated J774A.1 macrophage cells; PA-LF: J774A.1 macrophage cells incubated 3.5 h with LF (1.1 nM) and PA83 (6 nM); MAP2 and MAP3: J774A.1 macrophage cells incubated 3.5 h with LF (1.1 nM) and PA83 (6 nM) in the presence of anti-PA63 MAP peptides at two concentrations (8 e 0.8 mM).

FIG. 3: IC50 evaluation of MAP2 and MAP3.

(a) Inhibitory activity of MAPs on PA63-LF binding in ELISA. Purified oligomeric PA63 was incubated in LF-coated wells, in the presence of MAPs.

(b) Inhibitory activity of MAPs on lethal-toxin-induced cell mortality. J774A.1 cells were incubated with purified PA83 and LF in the presence of MAPs. Assays were performed in triplicate and half-maximal inhibition IC₅₀ was calculated by non-linear regression analysis using GraphPad Prism 3.02 software.

FIG. 4: Improvement of MAP activity.

(a) Alanine scanning of MAP3 sequence. 12 different tetra-branched MAPs, each carrying a single alanine substitution, were tested for inhibition of PA63-LF binding in ELISA and compared with MAP3 activity in the same experiment. MAPs were tested at 200-fold and 50-fold molar excess with respect to PA63 concentration. Results are from triplicate samples in the two experiments.

(b) Summary of inhibitory activity of MAPs derived from progressive shortening of MAP2 (left panel) and MAP3 (right panel) sequences on PA63-LF binding in ELISA. Results are from at least three experiments for each peptide.

≦peptide activity is within or below the SD of its original lead peptide tested under the same conditions; <; peptide activity is below the SD of its original lead peptide; X; peptide has no inhibitory activity; ≧peptide activity is within or over the SD of its original lead peptide.

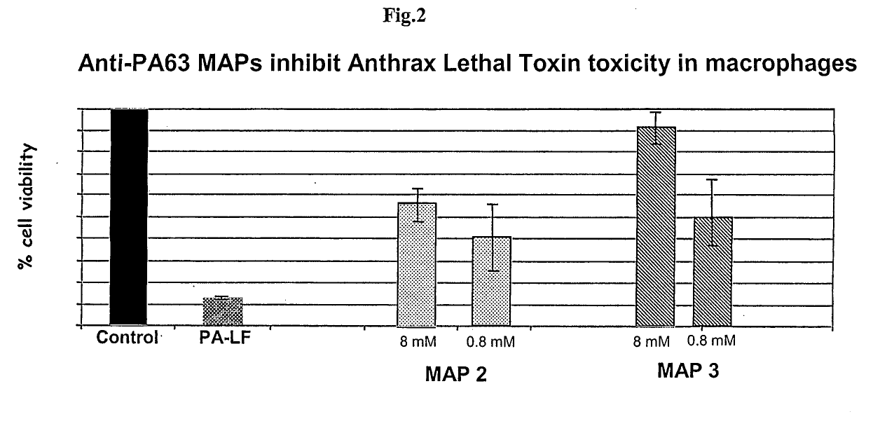

(c) Inhibitory activity of lead and modified MAPs on PA63-LF binding in ELISA. Results are from three independent experiments for each peptide, performed as described in FIG. 2a.

FIG. 5 Inhibitory activity of the best 7-mer MAPs on PA63-LF binding in ELISA. Bar colors correspond to the different MAP/PA ratio indicated in the legend.

FIG. 6 Inhibitory activity of lead and modified MAPs on lethal-toxin-induced cell death.

(a) J774A.1 cells were incubated with purified PA83 and LF in the presence of MAPs at concentrations of 8 μM (light grey bars) and 0.8 μM (dark grey bars) concentration Results are from triplicate samples in three independent experiments.

(b) Comparison of MAP3 and MAP3V/A inhibitory activity. Assays were performed in triplicate and IC₅₀ were calculated by nonlinear regression analysis using GraphPad Prism 3.02 software. Dotted line shows average cell viability obtained in the absence of peptides.

FIG. 7 Kinetics of PA63 binding. Overlay of sensorgrams from BIACORE analysis of PA63 (20 μg/ml) binding to biotinylated monomeric peptides p2 (TLPYWWLTPSNP) (Seq.Id.2), p3 (NVMTYWWLDPPL) (Seq.Id.3) and YWWLTPPP (Seq.Id.4) bound to a SA sensor chip.

FIG. 8 Table describing data from in-vivo experiments in rats. CONTROL: Rats injected with PA83 (40 μg) and LF (8 μg).

### DESCRIPTION OF THE INVENTION

Within the scope of the present invention, amino acid sequences are indicated with the one-letter amino acid acronyms, according to IUPAC-IUB rules.

The experimentation described below was conducted starting from a commercial library of peptides with random sequence in which each peptide is formed by 12 amino acid residues, from which specific peptides for PA63 were selected. Some of these peptides have been found to be particularly effective in inhibiting the binding between LF and PA, and were thus subjected to further characterisations and manipulations, described in the following experimental part, finally finding that the peptides comprising the sequence having aminoacidic formula from the amino terminal end YWWLX′P where X′ is any amino acid, and in particular it is chosen between T, D and A, are effective in blocking the interaction of PA with LF, i.e. the interaction of the two protein components which naturally form the lethal toxin of B. anthracis. Particularly effective were found the peptides having an amino acid sequence comprised in the following group: YWWLDPP, YWWLEPP, YWWLHPP, YWWLKPP, YWWLQPP, YWWLTPP (all included in Seq.Id. 22); TLPYWWLTPSNP (Seq.Id.2), NVMTYWWLDPPL (Seq.Id.3), YWWLTPPA (Seq.Id.5), YWWLTPPP (Seq.Id.4), YWWLTPPQ (Seq.Id.6), AVMTYWWLDPPL (Seq.Id.7), NAMTYWWLDPPL (Seq.Id.8), NVMTYWWLAPPL (Seq.Id.9), TLAYWWLTPSNP (Seq.Id.10), NVMTYWWLDPPA (Seq.Id.11), NDMTYWWLDPPL (Seq.Id.12), NEMTYWVADPPL (Seq.Id.13), NGTMYWWLDPPL (Seq.Id.14), NNMTYWWLDPPL (Seq.Id.15), NPMTYWWLDPPL (Seq.Id.16), NQMTYWWLDPPL (Seq.Id.17), NSMTYWWLDPPL (Seq.Id.18), NTMTYWWLDPPL (Seq.Id.19) and NYMTYWWLDPPL (Seq.Id.20).

The linear peptides according to the invention can be exposed on biological surfaces, e.g. on membranes of bacterial and/or eukaryotic and virus cells. The aforesaid peptides were then synthesised both in the linear form and in the branched MAP (Multiple Antigen Peptide) form, having formula (I)

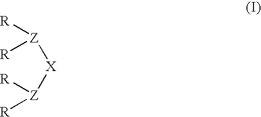

where:
R is a linear peptide as defined above,
X is a bifunctional molecule;
Z is selected between X and the group (II)

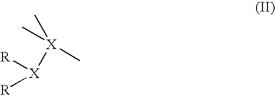

in which X and R are as defined above.

According to a preferred embodiment of the invention, Z is X, so that the resulting peptides in MAP form have a tetra ramified structure.

In the MAP peptides according to the invention, the bifunctional molecule X is, for example, an amino acid having at least two functional aminic groups, and it is preferably chosen in group consisting of lysine, ornithine, nor-lysine and amino alanine. Other embodiments of the MAP peptides of the invention are those in which in the formula (I) X is chosen in the group consisting of aspartic acid and glutamic acid, or X is chosen in the group consisting of propylene glycol, succinic acid, diisocyanates or diamines.

The peptides according to the present invention, be they in linear form or in MAP form, can be used effectively as laboratory reactants for identifying the Protective Antigen PA secreted by B. anthracis, which may be present in material for human and/or veterinary use, and also for preparing pharmaceutical compositions useful as antidotes in the intoxication caused by the Lethal Toxin of Bacillus anthracis.

Such pharmaceutical compositions comprising as an active ingredient at least one peptide in linear form or in MAP form may also comprise pharmaceutically acceptable excipients and/or diluents, commonly used in pharmaceutical formulations.

The synthesis of peptides in this form offers several advantages. MAPs are stable to peptidases and proteases of biological fluids, which makes them more suitable for new drug development¹⁶. Another advantage is that synthesis in MAP form of peptide sequences selected from a phage library, enables the activity of phage peptides to be retained. This may be due to some similarity between the structural arrangements of peptides in the MAP and in the phage-exposed forms. In a MAP molecule, peptide sequences are linked to the lysine core by their C-terminus, as when they are expressed on the phage fusion protein. Moreover, since MAPs contain more peptide copies, they enable multivalent binding, increasing binding efficiency, like in phage-peptides.

### Experimental Data

### Expression and Purification of PA

Recombinant PA was produced with a C-terminal hexa-histidine tag, and purified by Ni-chelate affinity chromatography. The expression vector pet20b+ containing the recombinant PA gene was transformed into the E. coli BL21 (DE3) cells. Cultures were grown in 2XTY medium (Oxoid) with 0.2% glucose and 100 μg/ml ampicillin at 37° C. to an OD₆₀₀ of 0.8, and protein expression was induced by 1 mM isopropyl-□-D-thiogalactopyranoside (IPTG, Inalco) for 18 h at 30° C. The bacterial suspension was centrifuged at 12,000×g for 30 min at 4° C. The pellet was resuspended in a solution of 50 mM HEPES-NaOH (pH 7.5), 0.5 M NaCl, 1 mM PMSF, 5 mM DTT containing 0.35 mg/ml lysozyme, then incubated for 30 min at room temperature. Triton X-100 was added to a final concentration of 1% and this was followed by ultrasound sonication in 5 bursts of 30 sec. The extract was treated with Dnase I (100 IU) in the presence of 4 mM MgCl₂ for 1 h at room temperature. The inclusion bodies were sedimented by centrifugation at 30,000×g for 30 min at 4° C. The pellet was washed twice with PBS (pH 7.4) containing 1% triton X-100 followed by spinning at 30,000×g for 30 min at 4° C. The pellet was then solubilized in 50 mM HEPES-NaOH (pH 7.5), 6 M guanidine HCl, 25 mM DTT and left for 1 h at 4° C. Insoluble material was removed by centrifugation at 100,000×g for 10 min. The solubilized proteins were diluted 1:10 into cold folding buffer composed of 50 mM HEPES (pH 7.5), 0.2 M NaCl, 1 mM DTT, 1 M NDSB201 [3(1-pyridinio)-1-propane sulfonate] (Calbiochem) and incubated for 1 h at 4° C. The protein solution was dialyzed overnight against 15 mM imidazole in 50 mM NaH₂PO₄, 500 mM⁻NaCl (pH 7.4) at 4° C. and loaded onto a Ni-NTA agarose column (Quiagen). The column was washed, and the protein was eluted with 100 mM imidazole in 50 mM NaH₂PO₄, 500 mM NaCl (pH 7.4) according to the manufacturer's protocol. The protein was then dialyzed overnight against PBS (pH 7.4) at 4° C. The purity of PA was evaluated by SDS-polyacrylamide gel electrophoresis and Coomassie Blue staining.

### Preparation of Nicked PA (PA63)

Nicked PA was produced by treatment of purified recombinant PA with trypsin at a final trypsin/PA ratio of 1:1000 (wt/wt) for 45 min at room temperature. The reaction was stopped by adding soybean trypsin inhibitor at a final trypsin/inhibitor ratio of 1:20 (wt/wt). Oligomeric PA63 was prepared from activated PA by purification on HiTrap Q FF column (Amersham Biosciences) in 20 mM Tris-HCl (pH 8.0) with a 0-1 M NaCl gradient. The protein was then dialyzed overnight against PBS (pH 7.4) at 4° C. The purity of PA63 was checked by SDS-polyacrylamide gel electrophoresis and Coomassie Blue staining.

### Expression and Purification of LF

Fusion protein of glutathione S-transferase (GST) and LF was expressed in E. coli strain BL21 (DE3) transformed with the expression vector pGEX(2TK) (Amersham Biosciences) containing the LF gene. Fusion protein expression was achieved by pre-inoculating a single bacterial colony in 15 ml 2XTY and 100 μg/ml ampicillin at 37° C. overnight. This culture was diluted 1:200 and cultured in 2XTY and 100 μg/ml ampicillin at 37° C. to an OD₆₀₀ of 1. Protein expression was induced by addition of 0.1 mM isopropyl-β-D-thiogalactopyranoside (IPTG, Inalco) for 18 h at 30° C. Bacterial pellet was separated from supernatant and resuspended in 40 ml cold PBS (pH 7.4) with 1 mM PMSF and sonicated in 4 bursts of 1 min. The extract was treated with Dnase I (50 IU) in the presence of 4 mM MgCl₂ and 1% Triton X-100 in mild agitation for 30 min. The sample was centrifuged at 12,000×g for 30 min and the supernatant treated as follows.

Purification was performed by affinity chromatography on Glutathione Sepharose 4B (Amersham Biosciences) according to the manufacturer's instructions. LF was eluted adding 50 IU of thrombin (Amersham Biosciences) in PBS for each ml of Glutathione Sepharose bed volume and incubating for 3 h at room temperature. The eluted solution was then collected and dialysed overnight against 20 mM Tris-HCl (pH 8.0) at 4° C. followed by anion exchange chromatography using a fast performance liquid chromatography (FPLC) system, which was performed to remove thrombin or to concentrate protein. Briefly, 3.5 ml of a 0.68 mg/ml solution of LF were loaded on a 1 ml Resource Q column (Amersham Biosciences) to obtain 1 ml of 1.5 mg/ml protein solution by eluting with 0.5 M NaCl in 20 mM Tris-HCl (pH 8.0). The right molecular weight protein was then separated from the proteolytic fragments by FPLC, using a size exclusion column (Superdex 200, 10/300 GL, Amersham Biosciences) pre-equilibrated with 20 mM Tris-HCl (pH 8.0). The eluted protein was newly concentrated using a Resource Q column (as above) to obtain 0.5 ml of protein at 0.45 mg/ml. The protein was finally dialyzed overnight against PBS (pH 7.4) at 4° C. The purity (more than 90%; not shown) of LF was then tested by SDS-polyacrylamide gel electrophoresis and Coomassie Blue staining.

### Selection of Anti-PA 63 Peptides

A random 12mer peptide phage library Ph.D.12 (New England Biolabs, USA) was panned on the proteolytically activated form of anthrax protective antigen (PA63). Microtiter wells were coated overnight at 4° C. with 100 μl purified PA63 (20 μg/ml) in 0.1 M NaHCO₃ (pH 8.6) and blocked with 0.1 M NaHCO₃ (pH 8.6)-0.5% BSA. In the first round of biopanning, 10 μl phage library [4×10¹⁰ plaque-forming units (pfu)] was added to the wells and allowed to bind for 1 h at room temperature with gentle rocking. After washing 10 times with TBS [50 mM Tris-HCl (pH 7.5), 150 mM NaCl]-Tween 20 0.1%, bound phages were eluted by competition using 100 μl of 200 μg/ml purified LF with gentle rocking at room temperature for 1 h.

The eluted phages were amplified in 20 ml mid-log phase E. coli ER2738 (OD₆₀₀=0.5) at 37° C. with vigorous shaking for 4.5 h. The cells were pelleted at 6,000×g at 4° C. for 10 min, the supernatant was removed to a fresh tube and re-spun briefly. The upper 80% of the supernatant containing phages was moved to a fresh tube with 1/6 volume 20% (wt/vol) PEG-8000 in 2.5 M NaCl and incubated at 4° C. for 1 h. The phage particles were isolated by centrifugation and the phage pellet was resuspended in approximately 1 ml TBS, respun briefly and re-precipitated with 1/6 volume PEG-NaCl by incubation at 4° C. for 60 min. The precipitated phages were re-centrifuged and the pellet was resuspended in 200 μl TBS. The isolated phage particles were finally centrifuged for 1 min to eliminate any remaining insoluble matter. The amplified phages were titered on LB medium-IPTG-X-Gal plates to ensure an input volume corresponding to 1-2×10¹¹ pfu and subsequently subjected to two further rounds of biopanning as described above, except that washing was performed by increasing the Tween 20 concentration from 0.1% to 0.5%. The phage fractions in all biopanning processes were titred to determine the degree of selection.

The binding of selected clones to PA63 after the third round of panning was evaluated by Phage-ELISA on 96-well microtitre plates coated overnight at 4° C. with 100 μl 20 μg/ml purified PA63.

Each phage clone was used to infect 250 μl of E. coli ER2738 diluted 1:100 from an overnight culture in LB medium. Single clones were propagated with gentle agitation at 37° C. for 4.5 h. The amplified phages were isolated by centrifugation and incubated for 1 h at 37° C. in PA63-coated 96 microwell plate previously blocked with PBS-3% BSA (pH 7.4) for 1 h. Then, anti-M13 peroxidase conjugate-antibody (Amersham) was incubated at a 1:2,500 dilution in PBS-3% BSA for 1 h at 37° C. After further washing, antibody binding was detected using Tetramethyl benzidine (TMB) substrate and the reaction was stopped with H₂SO₄. The plate was read at 450 nm (corrected for absorption at 655 nm) using a microplate reader.

### Peptide Synthesis

Monomeric peptides were synthesized as peptide amide by an automated synthesizer (MultiSynTech, Witten, Germany) on a Rink Amide MBHA resin (Nova Biochem) using 9-fluorenylmetlhoxycarbonyl chemistry and O-(benzotriazol-1-yl)-N,N,N′,N′-tetramethyluronium hexafluorophosphate/1,3-diisopropylethylamine activation. MAPs were synthesized on Fmoc₄-Lys₂-Lys-βAla Wang resin. Side chain protecting groups were tert-butyl ester for E; trityl for Q; tert-butoxycarbonyl for K and W; 2,2,4,6,7-pentamethyldihydrobenzofuran-5-sulfonyl for R; and tert-butyl ether for S. Peptides were then cleaved from the resin and deprotected by treatment with trifluoroacetic acid containing water and triisopropylsilane (95/2.5/2.5). Crude peptides were purified by reversed-phase chromatography on a Vydac C18 column. Identity and purity of final products was confirmed by Ettan™ MALDI-TOF mass spectrometry (MS) (Amersham Biosciences).

### PA-LF Binding (ELISA)

96-well microtiter plates (Nunc) were coated with 5 μg/ml LF in PBS overnight at 4° C. After PBS washing, plates were blocked with PBS-3% BSA for 1.5 h at 37° C. Wells were then incubated with PA63 (5 μg/ml) alone or with anti-PA63 peptides at several concentrations, for 1 h at 37° C. After washing with PBS-0.05% Tween and PBS the plate was incubated with anti-his₆-peroxidase (Roche) diluted 1:500 in PBS-3% BSA for 45 min at 37° C. Binding was detected using TMB substrate and the reaction was stopped with H₂SO₄. The plate was read at 450 nm (corrected for absorption at 655 nm) using a microplate reader.

### Cell Culture

The J774A.1 mouse macrophage-like cell line (ATCC) was maintained in RPMI 1640 medium supplemented with 10% heat-inactivated fetal bovine serum and antibiotics (100 IU/ml penicillin and 100 μg/ml streptomycin) at 37° C. in a humidified atmosphere of 5% CO₂.

### Cytotoxicity Assay

J774A.1 cells were harvested by scraping, plated at a density of 2.5×10⁴ in 200 μl/well in a 96-well culture plate and allowed to adhere by incubation for 18 h at 37° C. in a humidified atmosphere of 5% CO₂. The next day, medium was removed and replaced with RPMI (100 μl/well) containing 0.5 μg/ml recombinant PA and 0.1 μg/ml recombinant LF with or without anti-PA63 MAPs. Cell viability was determined after 3.5 h incubation using the 3-[4,5-dimethylthiazol-2-yl]-2,5-diphenyltetrazolium bromide (MTT) dye assay. MTT was added to the cells to a final concentration of 0.5 mg/ml and the cells were incubated for 1.5 h at 37° C. to allow uptake and oxidation of the dye by viable cells. The cells were then solubilized with 100 μl/well of 10% sodium dodecyl sulfate (SDS), 45% dimethylformamide (pH 4.5) and the absorbance was measured at 595 nm (corrected for absorption at 655 nm) using a microplate reader.

### BIACORE

All experiments were performed on a BIACORE 1000 Upgraded and all materials were purchased by Biacore International AB, Uppsala, Sweden.

Biotinylated monomeric peptides, diluted at 100 μg/ml in Hepes Buffer Saline (10 mM Hepes, 150 mM NaCl, 3.4 mM EDTA, 0.005% polysorbate 20, pH 7.4) (HBS), were immobilized on streptavidin-coated SA sensor chip at the flow rate of 10 μl/min. For kinetic experiments serial dilutions of PA63, diluted in HBS at concentration ranging from 1 μM to 10 nM, were injected at a flow rate of 10 μl/min over immobilized peptides.

Association and dissociation kinetic rate constants (kon and koff) and the equilibrium association constant KA were calculated using the BIAevaluation 3.0 software.

### Inhibition of Anthrax Lethal Toxin Activity In Vivo

Animal procedures were approved by the Ethical Committee of the Azienda Ospedaliera Universitaria Senese on 28 May 2004. Fisher 344 rats (average weight 250 g), were anesthetized by intraperitoneal injection of ketamine and acepromazine and inoculated in the tail vein with 40 μg purified recombinant PA and 8 μg purified recombinant LF diluted in PBS.

Peptide powder was resuspended in PBS to a concentration of 2 mg/ml, sonicated for 60 min, additioned with 0.3% 5 M NaOH, diluted to 1 mg/ml (pH 7.5) and injected in the tail vein of rats 5 min after the lethal toxin injection. In some cases a second injection of peptide inhibitor was given 1 hour after the first injection. When anthrax symptoms were obvious, the rats were killed to avoid unnecessary distress. Surviving rats were sacrificed after 7 days.

### EXAMPLES

### Example 1

### Selection of Peptide Inhibitors

Selection of peptides that interfere with PA/LF binding was carried out by incubating the large phage peptide library Ph.D.-12™ with purified recombinant PA63. Elution of specific anti-PA63 phage peptides was obtained by addition of recombinant LF which, by combining with PA63, selectively detach peptides bound to PA63 on its LF-binding site, leaving all other phages that recognize different PA63 regions, attached to the protein. This system resulted particularly efficient in our hands contrary to what published by others¹⁷.

All sequenced clones shared the consensus motif YWWLX′P (Seq.Id.1), though not always in the same sequence position. This short primary sequence is shared by previously described PA-binding peptides obtained from the same phage library by a different selection approach¹³ and is not present in the sequence of LF, EF or PA20. Two phage peptides giving the highest inhibition of PA63-LF binding in a competition phage ELISA and respectively carrying the sequences TLPYWWLTPSNP (p2) (Seq.Id.2) and NVMTYWLDPPL (p3) (Seq.Id.3), were chosen for chemical synthesis and further experiments.

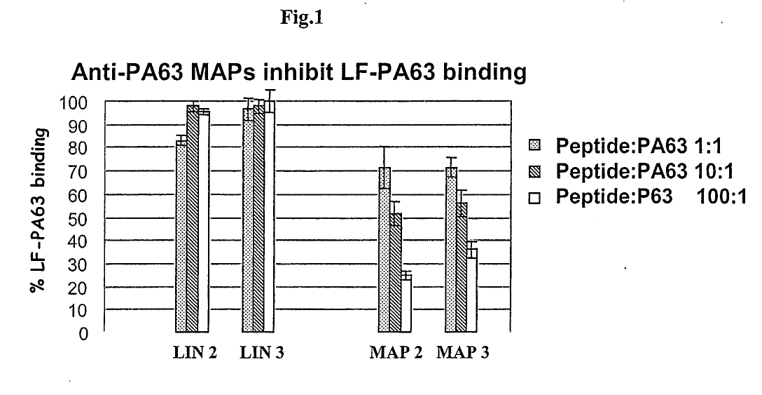

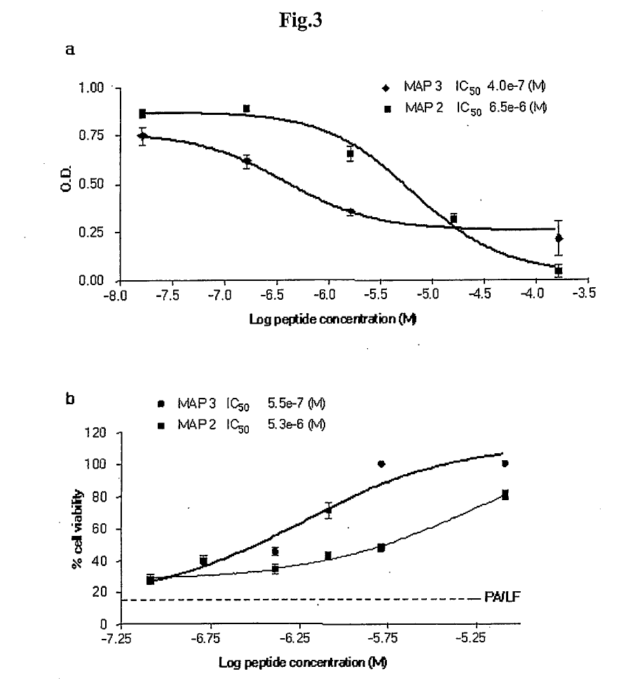

The two selected sequences were synthesized in monomeric and MAP forms. MAPs are dendrimeric molecules in which several peptide copies are synthesized on a lysine core¹⁴. In our case we synthesized tetra-branched MAP using a core of three lysine. Monomeric peptides did not significantly inhibit PA63-LF binding in ELISA (FIG. 1), even when used at up to 100-fold molar excess with respect to PA63. On the contrary, the MAPs also gave significant inhibition at a 1/1 molar ratio. The IC50 of MAP3 was about one log lower than that of MAP2 (FIG. 3a). The MAPs were also tested for their ability to inhibit murine macrophage cell death, induced by incubation with PA83 and LF. The two MAPs produced a significant reduction of lethal-toxin-induced cell death (FIG. 2). The resulting IC50 were comparable to those obtained in the test for inhibition of PA63-LF binding (FIG. 3b).

MAP2 and MAP3 both specifically bound PA63 and did not bind purified recombinant PA83 or LF in ELISA (not shown).

### Example 2

### Modification of Lead Sequences

In order to gain further information about peptide sequence-activity correlation to be used for enhancing MAP efficiency, the sequence of the two lead peptides were systematically modified by alanine-scanning and progressive sequence shortening, followed by random re-elongation. All peptides were always in the tetra-branched MAP form.

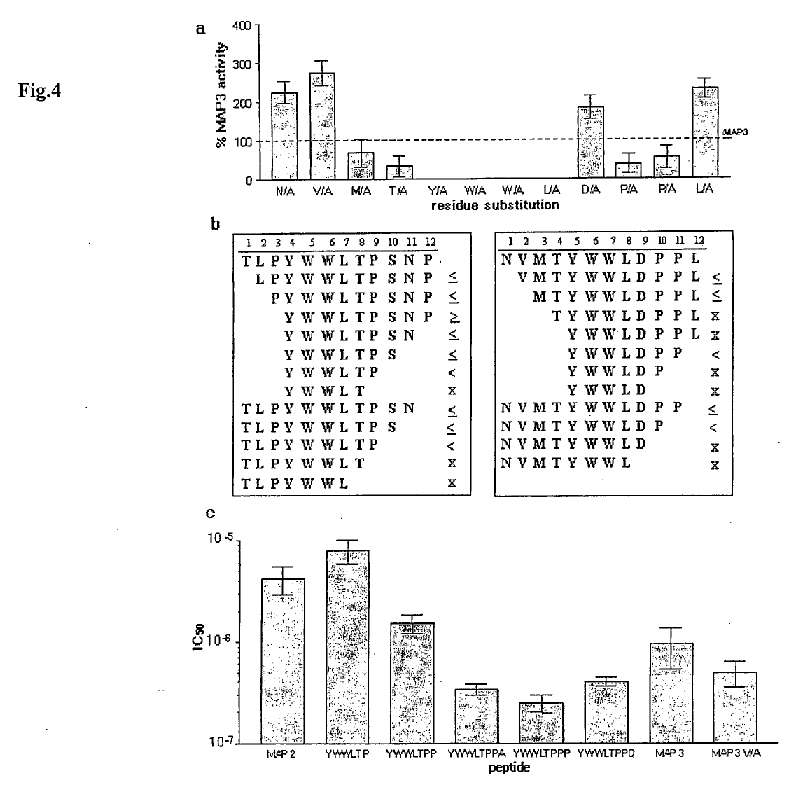

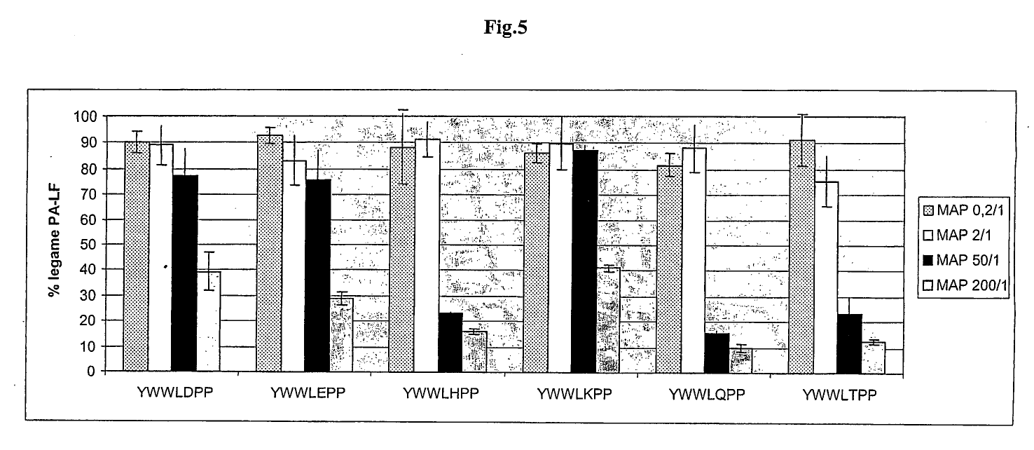

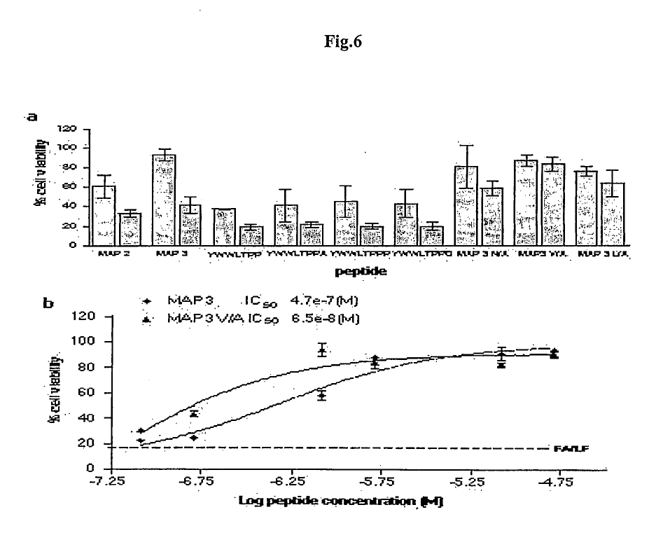

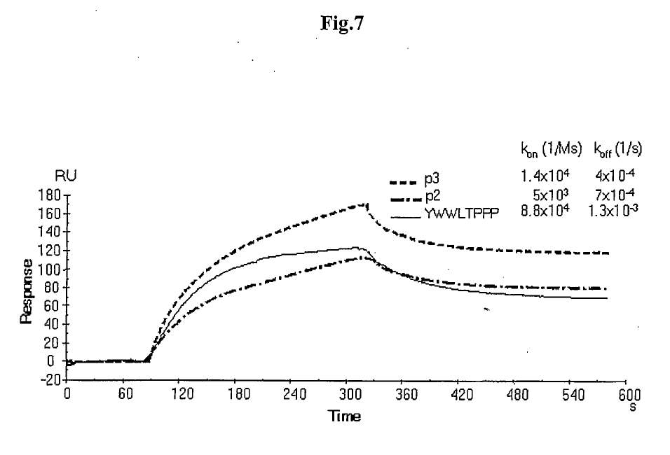

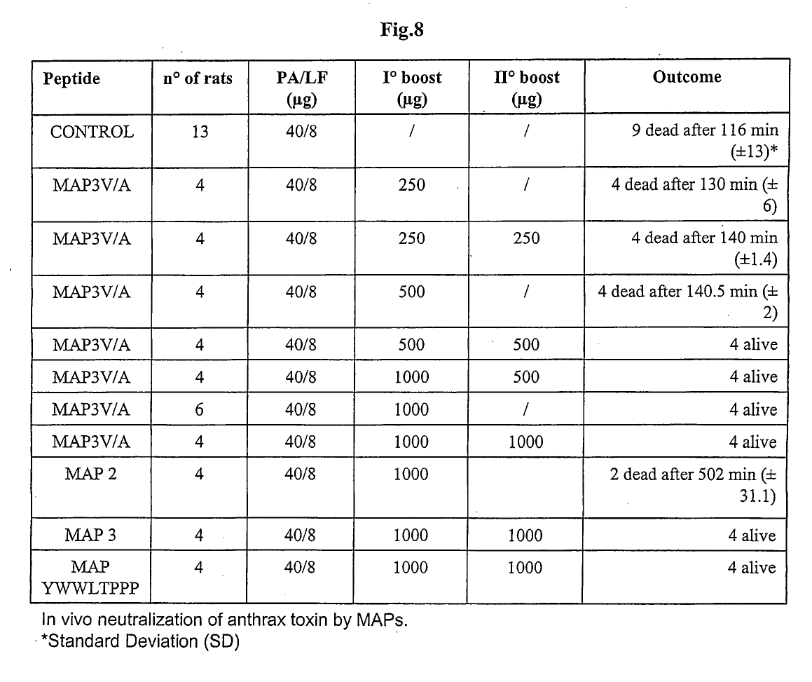

Alanine scanning of MAP3 sequence allowed identifying different critical position for peptide activity. The substitution of any residue inside the YWWLX′P (Seq.Id.1) consensus sequence completely abolished peptide inhibitory activity (FIG. 4a). Moreover, substitution of metionine and threonine respectively in position 3 and 4, and proline in position 10 and 11, all brought to loss of peptide activity. The same alanine scanning led to selection of four new peptides AVMTYWWLDPPL (Seq.Id.7), NAMTYWWLDPPL (Seq.Id.8), NVMTYWWLAPPL (Seq.Id.9) and NVMTYWWLDPPA (Seq.Id.11)) with better efficiency in inhibiting binding of PA63 to LF in ELISA.

The substitution of the amino acid in position 2 of the MAP 3 with any other residue brought also to the identification of 9 new peptides NDMTYWWLDPPL (Seq.Id.12), NEMTYWWLDPPL (Seq.Id.13), NGMTYWWLDPPL (Seq.Id.14), NNMTYWWLDPPL (Seq.Id.15), NPMTYWWLDPPL (Seq.Id.16), NQMTYWWLDPPL (Seq.Id.17), NSMTYWWLDPPL (Seq.Id.18), NTMTYWWLDPPL (Seq.Id.19) and NYMTYWWLDPPL (Seq.Id.20), with a good efficiency in inhibiting binding of PA63 to LF in ELISA.

All 12 new branched peptides carrying one single alanine substitution, starting from the sequence of peptide MAP2, when tested for PA-LF inhibition in ELISA, produced a slight decrease in inhibitory activity with respect to the lead peptide. Substitution of each residue in the YWWLX′P (Seq.Id.1) sequence with Ala again led to complete loss of peptide activity (not shown).

Shortening the MAP3 sequence by progressive deletion of single residues at the N- or C-terminus, indicated that deletion of residues in positions 1, 2 and 12 produced a limited decrease in inhibitory activity. Further deletions produced a sharp decrease in peptide activity (FIG. 4b).

Similar shortening of the MAP2 sequence revealed the minimal sequence able to inhibit PA-LF binding in ELISA (FIG. 4b). This six residue sequence (YWWLTP) (included in Seq.Id.4) was extended by systematically adding all natural amino acids (not including cysteine, to avoid possible inter and intra-peptide bonds in the MAP molecule) at both termini. Extension at the N-terminus did not produce better peptides in terms of inhibition of PA63-LF binding. On the contrary, extension at the C-terminus led to selection of peptide YWWLTPP (included in Seq.Id.4), which has superior inhibitory efficiency. Peptides having lysine and arginine in position 7 were completely inactive. Further elongation of peptide YWWLTPP (included in Seq.Id.4), revealed sequences YWWLTPPA (Seq.Id.5), YWWLTPPQ (Seq.Id.6) and YWWLTPPP (Seq.Id.4) with even better efficiency in inhibiting PA63-LF binding (FIG. 4c). Addition of positively charged residue in position 8, again resulted in peptides with no inhibitory activity. Interestingly, these 8mer peptides share a PPX sequence at the C-terminus, which is present in the 12mer peptides with higher activity (MAP3 and peptides derived from this lead sequence) and not in other lower affinity peptides (MAP2). The presence of these two prolines close to the YWWLX′P (Seq.Id.1) motif, might increase peptide binding to PA63 and this could explain why the MAP2 sequence could be shortened at the C-terminus, up to the proline in position 9, whereas the MAP3 sequence could not. This hypothesis is also confirmed by alanine scanning of MAP3, which showed that substitution of proline 11 and particularly proline 10, resulted in a clear decrease in peptide activity, whereas substitution of the aspartic acid following the YWWLX′P (Seq.Id.1) consensus produced a peptide with increased activity. Moreover, alanine scanning of the minimum inhibiting sequence YWWLTP (included in Seq.Id.4) confirmed that the only residue that could be substituted with no decrease in activity was threonine (not shown).

From our results, the consensus sequence YWWLX′PPX″ (Seq.Id.21) can be derived, which may enable high affinity binding to PA63. Moreover, we demonstrated that positively charged residues cannot be inserted in position 8 of this consensus and leucine cannot be inserted in position 8, when position 5 is aspartic acid. In order to define the best residue to be introduced in position 5 of the consensus peptide sequence YWWLX′PP (Seq.Id.22), a further sublibrary of MAPs was synthesized. Inhibition of PA-LF binding in ELISA by the 19 resulting MAPs showed that peptides with D, E, H, K Q and T residues in that position efficiently interfere with PA-LF binding (FIG. 5)

All these second generation branched peptides, were also compared with the lead peptides MAP2 and MAP3 for their ability to inhibit cell mortality, induced by incubation with PA83 and LF. Unlike what we found in experiments for inhibition of PA63-LF binding in ELISA, short peptides turned out to be less active than 12mer peptides in inhibiting cell mortality (FIG. 6a). Among 12 mer peptides the MAP3V/A had an IC50 for inhibition of PA83/LF-induced cell mortality that was one log lower than that of MAP3 (FIG. 6b).

### Example 3

### Binding Kinetics of 8mer and 12mer Peptides

Inhibitory activity of MAPs as measured in ELISA does not give any indication of possible differences in kinetic rates that might be related to differences in peptide length or sequence. The kinetics of binding of monomeric peptides p2, p3 and the 8mer peptide YWWLTPPP (Seq.Id.4) to PA63 was therefore analysed by BIACORE (FIG. 7). Comparison of equilibrium constants (KA) of peptide-PA63 binding essentially confirmed the IC50 ratios of corresponding MAPs, as calculated in PA63-LF binding experiments in ELISA (FIG. 4c). The KA of the 8mer peptide YWWLTPPP (Seq.Id.4) was 6.8×10⁷ (1/M), about 10-fold higher than that of the lead 12mer p2 (7.2×10⁶, 1/M) and slightly better than that of the 12mer peptide p3 (3.4×10⁷, 1/M). Analysis of kinetic rates indicated that, although the increase in KA of the 12mer peptide p3 with respect to the 12mer peptide p2 is due to an almost equally distributed improvement in both kon and koff (kon were 5×10³, 1/Ms and 1.4×10⁴, 1/Ms; koff were 7×10⁻⁴, 1/s and 4×10⁻⁴, 1/s, for p2 and p3 respectively) improvement of 8mer peptide KA with respect to 12 mer peptides was mainly due to an increase in kon (8.8×10⁴, 1/Ms), whereas the koff (1.3×10⁻³, 1/s) of the 8mer peptide was faster than that of both 12mer peptides.

### Example 4

### In Vivo Neutralization of Anthrax Lethal Toxin

Experiments were carried out with the peptides MAP 2, MAP 3, MAP3V/A and MAP YWWLTPPP (Seq.Id.4). Fisher 344 rats were injected with a mixture containing 40 μg of purified PA83 and 8 μg of purified LF. Under these conditions rats died in about two hours. 5 minutes after injection of Lethal toxin, rats were injected with the peptide inhibitor. In some experiments rats received a second boost of peptide, one hour after injection of the toxin. Injection of MAP 3, MAP YWWLTPPP (Seq.Id.4) and MAP3V/A, this latter either in a single boost of 1 mg or in two boosts of 500□g each, completely neutralized toxicity (FIG. 8).

### REFERENCES

1. Bradley, K. A., Mogridge, J., Mourez, M., Collier, R. J. & Young, J. A. Identification of the cellular receptor for anthrax toxin. Nature 414, 225-229 (2001).
2. Chauhan, V. & Bhatnagar, R. Identification of amino acid residues of anthrax protective antigen involved in binding with lethal factor. Infect. Immun. 70, 4477-4484 (2002).
3. Kurzchalia T. Anthrax toxin rafts into cells. J. Cell Biol. 160, 295-296 (2003).
4. Leppla, S. H. Anthrax toxin edema factor: a bacterial adenylate cyclase that increases cyclic AMP concentrations of eukaryotic cells. Proc. Natl. Acad. Sci. USA 79, 3162-3166 (1982).
5. Duesbery, N. S. et al. Proteolytic inactivation of MAP-kinase-kinase by anthrax lethal factor. Science 280, 734-737 (1998).
6. Dixon, T. C., Meselson, M., Guillemin, J. & Hanna, P. C. Anthrax. N. Engl. J. Med. 341, 815-826 (1999).
7. Maynard, J. A. et al. Protection against anthrax toxin by recombinant antibody fragments correlates with antigen affinity. Nat. Biotechnol. 20, 597-601 (2002).
8. Wild, M. A. et al. Human antibodies from immunized donors are protective against anthrax toxin in vivo. Nature Biotechnol. 11, 1305-1306 (2003).
9. Sellman, B. R., Mourez, M. & Collier, R. J. Dominant-negative mutants of a toxin subunit: an approach to therapy of anthrax. Science, 292, 695-697 (2001).
10. Panchal, R. G. et al. Identification of small molecule inhibitors of anthrax lethal, factor. Nat. Struct. Mol. Biol. 11, 67-72 (2004).
11. Dell'Aica, I. et al., Potent inhibitors of anthrax lethal factor from green tea. EMBO Rep. 5, 418-422 (2004).
12. Sarac, M. S., Peinado, J. R., Leppla, S. H. & Lindberg, I. Protection against anthrax toxemia by hexa-D-arginine in vitro and in vivo. Infect. Immun. 72, 602-605 (2004).
13. Mourez, M. et al. Designing a polyvalent inhibitor of anthrax toxin. Nat. Biotechnol. 19, 958-961 (2001).
14. Tam, J. P. Synthetic peptide vaccine design: synthesis and properties of a high-density multiple antigenic peptide system. Proc. Natl. Acad. Sci. USA. 85, 5409-5413 (1988).
15. Lozzi, L. et al. Rational design and molecular diversity for the construction of anti-alpha-bungarotoxin antidotes with high affinity and in vivo efficiency. Chem. Biol. 10, 411-417 (2003).
16. Bracci, L. et al. Synthetic peptides in the form of dendrimers become resistant to protease activity. J. Biol. Chem. 278, 46590-46595 (2003).
17. Mourez, M. & Collier, R. J. Use of phage display and polyvalency to design inhibitors of protein-protein interactions. Methods Mol. Biol. 261, 213-228 (2004).

1.-20. (canceled)

21. Peptide comprising the amino acid sequence NH2-YWWLXT-COOH (Seq.Id.1), were X′ is an amino acid, and pharmaceutical acceptable salts thereof.

22. Peptide as claimed in claim 1, wherein X′ is an aminoacid chosen in the group consisting of T, D, A, E, H, K and Q.

23. Peptide according to claim 1 comprising the amino acid sequence NH2-YWWLX′PPX″-COOH (Seq.Id.21), were X′ and X″ are amino acids, and pharmaceutical acceptable salts thereof.

24. Peptide as claimed in claim 3, wherein X″ is an amino acid chosen in the group consisting of P, A and Q.

25. Peptide as claimed in claim 1, having an amino acid sequence comprised in the following group: YWWLDPP, YWWLEPP, YWWLHPP, YWWLKPP, YWWLQPP, YWWLTPP (all included in Seq.Id. 22); TLPYWWLTPSNP (Seq.Id.2), NVMTYWWLDPPL (Seq.Id.3), YWWLTPPA (Seq.Id.5), YWWLTPPP (Seq.Id.4), YWWLTPPQ (Seq.Id.6), AVMTYWWLDPPL (Seq.Id.7), NAMTYWWLDPPL (Seq.Id.8), NVMTYWWLAPPL (Seq.Id.9), TLAYWWLTPSNP (Seq.Id.10), NVMTYWWLDPPA (Seq.Id.11), NDMTYWWLDPPL (Seq.Id.12), NEMTYWWLDPPL (Seq.Id.13), NGMTYWWLDPPL (Seq.Id.14), NNMTYWWLDPPL (Seq.Id.15), NPMTYWWLDPPL (Seq.Id.16), NQMTYWWLDPPL (Seq.Id.17), NSMTYWWLDPPL (Seq.Id.18), NTMTYWWLDPPL (Seq.Id.19) and NYMTYWWLDPPL (Seq.Id.20).

26. Peptide as claimed in claim 21, exposed on biological surfaces.

27. Peptide as claimed in claim 22, exposed on biological surfaces.

28. Peptide as claimed in claim 23, exposed on biological surfaces.

29. Peptide as claimed in claim 24, exposed on biological surfaces.

30. Peptide as claimed in claim 25, exposed on biological surfaces.

31. Peptide as claimed in claim 26, wherein said biological surfaces are chosen among membranes of bacterial and/or eukaryotic and virus cells.

32. Peptide as claimed in claim 21, multimerised on a skeleton of polyacrylamide, on a skeleton of dextrane units or on a skeleton of ethylene glycol units.

33. Peptide in the form of Multiple Antigenic Peptides (MAP) having formula (I): 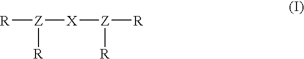
where:
R is a linear peptide as defined in claims 1-5;
X is a bifunctional molecule;
Z is selected between X and the group 11 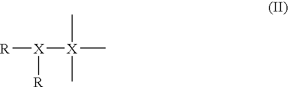
wherein X and R are as defined above; and pharmaceutically acceptable salts thereof.

34. The peptide as claimed in claim 33, wherein Z is X.

35. The peptide as claimed in claim 33, wherein X is an aminoacid having at least two functional aminic groups.

36. Peptide as claimed in claim 33, wherein X is chosen in the group consisting of lysine, ornithine, nor-lysine and amino alanine.

37. The peptide as claimed in claim 33, wherein X is an aminoacid chosen in the group consisting of aspartic acid and glutamic acid.

38. The peptide as claimed in claim 33, wherein X is an aminoacid chosen in the group consisting of propylene glycol, succinic acid, diisocyanates or diamines.

39. A method of preparing a pharmaceutical composition useful as antidotes in the intoxication from Lethal Toxin of Bacillus anthracis, comprising adding a peptide of the amino acid sequence NH2-YWWLXT-COOH (Seq.Id.1), were X′ is an amino acid, and pharmaceutical acceptable salts thereof, to a pharmaceutically acceptable carrier.

40. The method of claim 39, wherein X′ is an aminoacid chosen in the group consisting of T, D, A, E, H, K and Q.

41. The method of claim 39 comprising the amino acid sequence NH2-YWWLX′PPX″-COOH (Seq.Id.21), were X′ and X″ are amino acids, and pharmaceutical acceptable salts thereof.

42. The method of claim 41, wherein X″ is an amino acid chosen in the group consisting of P, A and Q.

43. The method of claim 39, having an amino acid sequence comprised in the following group: YWWLDPP, YWWLEPP, YWWLHPP, YWWLKPP, YWWLQPP, YWWLTPP (all included in Seq.Id. 22); TLPYWWLTPSNP (Seq.Id.2), NVMTYWWLDPPL (Seq.Id.3), YWWLTPPA (Seq.Id.5), YWWLTPPP (Seq.Id.4), YWWLTPPQ (Seq.Id.6), AVMTYWWLDPPL (Seq.Id.7), NAMTYWWLDPPL (Seq.Id.8), NVMTYWWLAPPL (Seq.Id.9), TLAYWWLTPSNP (Seq.Id.10), NVMTYWWLDPPA (Seq.Id.11), NDMTYWWLDPPL (Seq.Id.12), NEMTYWWLDPPL (Seq.Id.13), NGMTYWWLDPPL (Seq.Id.14), NNMTYWWLDPPL (Seq.Id.15), NPMTYWWLDPPL (Seq.Id.16), NQMTYWWLDPPL (Seq.Id.17), NSMTYWWLDPPL (Seq.Id.18), NTMTYWWLDPPL (Seq.Id.19) and NYMTYWWLDPPL (Seq.Id.20).

44. The method of claim 39, exposed on biological surfaces.

45. The method of claim 44, wherein said biological surfaces are chosen among membranes of bacterial and/or eukaryotic and virus cells.

46. The method of claim 39, wherein the peptide is multimerised on a skeleton of polyacrylamide, on a skeleton of dextrane units or on a skeleton of ethylene glycol units.

47. A method of preparing a pharmaceutical composition useful as antidotes in the intoxication from Lethal Toxin of Bacillus anthracis, comprising adding to a pharmaceutically acceptable carrier, a Peptide in the form of Multiple Antigenic Peptides (MAP) having formula (I): 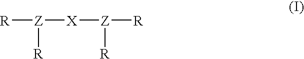
where:
R is a linear peptide as defined in claims 1-5;
X is a bifunctional molecule;
Z is selected between X and the group 11 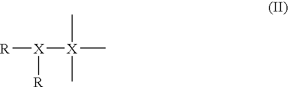
wherein X and R are as defined above; and pharmaceutically acceptable salts thereof.

48. The method of claim 47, wherein Z is X.

49. The method of claim 47, wherein X is an aminoacid having at least two functional aminic groups.

50. The method of claim 47, wherein X is chosen in the group consisting of lysine, ornithine, nor-lysine and amino alanine.

51. The method of claim 47, wherein X is an aminoacid chosen in the group consisting of aspartic acid and glutamic acid.

52. The method of claim 47, wherein X is an aminoacid chosen in the group consisting of propylene glycol, succinic acid, diisocyanates or diamines.

53. A method of preparing a laboratory reactant for the identification of the Protective Antigen PA secreted by B. anthracis, which may be present in material for human and/or veterinary use, comprising adding a peptide of the amino acid sequence NH2-YWWLXT-COOH (Seq.Id.1), were X′ is an amino acid, and pharmaceutical acceptable salts thereof, to a pharmaceutically acceptable carrier.

54. The method of claim 53, wherein X′ is an aminoacid chosen in the group consisting of T, D, A, E, H, K and Q.

55. The method of claim 53 comprising the amino acid sequence NH2-YWWLX′PPX″-COOH (Seq.Id.21), were X′ and X″ are amino acids, and pharmaceutical acceptable salts thereof.

56. The method of claim 55, wherein X″ is an amino acid chosen in the group consisting of P, A and Q.

57. The method of claim 53, having an amino acid sequence comprised in the following group: YWWLDPP, YWWLEPP, YWWLHPP, YWWLKPP, YWWLQPP, YWWLTPP (all included in Seq.Id. 22); TLPYWWLTPSNP (Seq.Id.2), NVMTYWWLDPPL (Seq.Id.3), YWWLTPPA (Seq.Id.5), YWWLTPPP (Seq.Id.4), YWWLTPPQ (Seq.Id.6), AVMTYWWLDPPL (Seq.Id.7), NAMTYWWLDPPL (Seq.Id.8), NVMTYWWLAPPL (Seq.Id.9), TLAYWWLTPSNP (Seq.Id.10), NVMTYWWLDPPA (Seq.Id.11), NDMTYWWLDPPL (Seq.Id.12), NEMTYWWLDPPL (Seq.Id.13), NGMTYWWLDPPL (Seq.Id.14), NNMTYWWLDPPL (Seq.Id.15), NPMTYWWLDPPL (Seq.Id.16), NQMTYWWLDPPL (Seq. Id.17), NSMTYWWLDPPL (Seq. Id.18), NTMTYWWLDPPL (Seq.Id.19) and NYMTYWWLDPPL (Seq.Id.20).

58. The method of claim 53, exposed on biological surfaces.

59. The method of claim 58 wherein said biological surfaces are chosen among membranes of bacterial and/or eukaryotic and virus cells.

60. The method of claim 53, wherein the peptide is multimerised on a skeleton of polyacrylamide, on a skeleton of dextrane units or on a skeleton of ethylene glycol units.

61. A method of preparing a laboratory reactant for the identification of the Protective Antigen PA secreted by B. anthracis, which may be present in material for human and/or veterinary use, comprising adding to an acceptable carrier, a Peptide in the form of Multiple Antigenic Peptides (MAP) having formula (I): 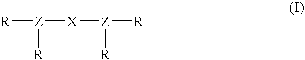
where:
R is a linear peptide as defined in claims 1-5;
X is a bifunctional molecule;
Z is selected between X and the group 11 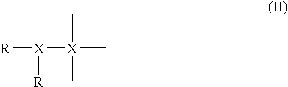
wherein X and R are as defined above; and pharmaceutically acceptable salts thereof.

62. The method of claim 61, wherein Z is X.

63. The method of claim 61, wherein X is an aminoacid having at least two functional aminic groups.

64. The method of claim 61, wherein X is chosen in the group consisting of lysine, ornithine, nor-lysine and amino alanine.

65. The method of claim 61, wherein X is an aminoacid chosen in the group consisting of aspartic acid and glutamic acid.

66. The method of claim 61, wherein X is an aminoacid chosen in the group consisting of propylene glycol, succinic acid, diisocyanates or diamines.

67. A pharmaceutical composition comprising as an active ingredient at least one peptide of the amino acid sequence NH2-YWWLXT-COOH (Seq.Id.1), were X′ is an amino acid, and pharmaceutical acceptable salts thereof, in combination with pharmaceutically acceptable excipients and/or diluents.

68. The composition of claim 67, wherein X′ is an aminoacid chosen in the group consisting of T, D, A, E, H, K and Q.

69. The composition of claim 67 comprising the amino acid sequence NH2-YWWLX′PPX″-COOH (Seq.Id.21), were X′ and X″ are amino acids, and pharmaceutical acceptable salts thereof.

70. The composition of claim 69 wherein X″ is an amino acid chosen in the group consisting of P, A and Q.

71. The composition of claim 67, having an amino acid sequence comprised in the following group: YWWLDPP, YWWLEPP, YWWLHPP, YWWLKPP, YWWLQPP, YWWLTPP (all included in Seq. Id. 22); TLPYWWLTPSNP (Seq.Id.2), NVMTYWWLDPPL (Seq.Id.3), YWWLTPPA (Seq.Id.5), YWWLTPPP (Seq.Id.4), YWWLTPPQ (Seq.Id.6), AVMTYWWLDPPL (Seq.Id.7), NAMTYWWLDPPL (Seq.Id.8), NVMTYWWLAPPL (Seq.Id.9), TLAYWWLTPSNP (Seq.Id.10), NVMTYWWLDPPA (Seq.Id.11), NDMTYWWLDPPL (Seq.Id.12), NEMTYWWLDPPL (Seq.Id.13), NGMTYWWLDPPL (Seq.Id.14), NNMTYWWLDPPL (Seq.Id.15), NPMTYWWLDPPL (Seq.Id.16), NQMTYWWLDPPL (Seq.Id.17), NSMTYWWLDPPL (Seq.Id.18), NTMTYWWLDPPL (Seq.Id.19) and NYMTYWWLDPPL (Seq.Id.20).

72. The composition of claim 67, exposed on biological surfaces.

73. The composition of claim 72 wherein said biological surfaces are chosen among membranes of bacterial and/or eukaryotic and virus cells.

74. The composition of claim 67, wherein the peptide is multimerised on a skeleton of polyacrylamide, on a skeleton of dextrane units or on a skeleton of ethylene glycol units.

75. A pharmaceutical composition, comprising a Peptide in the form of Multiple Antigenic Peptides (MAP) having formula (I): 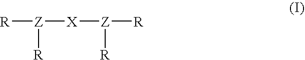
where:
R is a linear peptide as defined in claims 1-5;
X is a bifunctional molecule;
Z is selected between X and the group 11 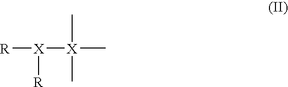
wherein X and R are as defined above; and pharmaceutically acceptable salts thereof.

76. The method of claim 75, wherein Z is X.

77. The method of claim 75, wherein X is an aminoacid having at least two functional aminic groups.

78. The method of claim 75, wherein X is chosen in the group consisting of lysine, ornithine, nor-lysine and amino alanine.

79. The method of claim 75, wherein X is an aminoacid chosen in the group consisting of aspartic acid and glutamic acid.

80. The method of claim 75, wherein X is an aminoacid chosen in the group consisting of propylene glycol, succinic acid, diisocyanates or diamines.


SEQUENCE LISTING (22 sequences)

SEQ ID NO: 1 - protein, 6 aa, Artificial sequence
  Synthetic from B. anthracis
YWWLXP

SEQ ID NO: 2 - protein, 12 aa, artificial sequence
  Synthetic from B. anthracis
TLPYWWLTPSNP

SEQ ID NO: 3 - protein, 12 aa, Artificial sequence
  Synthetic from B. anthracis
NVMTYWWLDPPL

SEQ ID NO: 4 - protein, 8 aa, Artificial sequence
  Synthetic from B. anthracis
YWWLTPPP

SEQ ID NO: 5 - protein, 8 aa, artificial sequence
  Synthetic from B. anthracis
YWWLTPPA

SEQ ID NO: 6 - protein, 8 aa, artificial sequence
  Synthetic from B. anthracis
YWWLTPPQ

SEQ ID NO: 7 - protein, 12 aa, Artificial sequence
  Synthetic from B. anthracis
AVMTYWWLDPPL

SEQ ID NO: 8 - protein, 12 aa, Artificial sequence
  Synthetic from B. anthracis
NAMTYWWLDPPL

SEQ ID NO: 9 - protein, 12 aa, Artificial
  Synthetic from B. anthracis
NVMTYWWLAPPL

SEQ ID NO: 10 - protein, 12 aa, Artificial
  Synthetic from B. anthracis
TLAYWWLTPSNP

SEQ ID NO: 11 - protein, 12 aa, Artificial
  Synthetic from B. anthracis
NVMTYWWLDPPA

SEQ ID NO: 12 - protein, 12 aa, Artificial
  Synthetic from B. anthracis
NDMTYWWLDPPL

SEQ ID NO: 13 - protein, 12 aa, Artificial
  Synthetic from B. anthracis
NEMTYWWLDPPL

SEQ ID NO: 14 - protein, 12 aa, Artificial
  Synthetic from B. anthracis
NGMTYWWLDPPL

SEQ ID NO: 15 - protein, 12 aa, Artificial
  Synthetic from B. anthracis
NNMTYWWLDPPL

SEQ ID NO: 16 - protein, 12 aa, Artificial
  Synthetic from B. anthracis
NPMTYWWLDPPL

SEQ ID NO: 17 - protein, 12 aa, Artificial
  Synthetic from B. anthracis
NQMTYWWLDPPL

SEQ ID NO: 18 - protein, 12 aa, Artificial
  Synthetic from B. anthracis
NSMTYWWLDPPL

SEQ ID NO: 19 - protein, 12 aa, Artificial
  Synthetic from B. anthracis
NTMTYWWLDPPL

SEQ ID NO: 20 - protein, 12 aa, Artificial
  Synthetic from B. anthracis
NYMTYWWLDPPL

SEQ ID NO: 21 - protein, 8 aa, Artificial
  Synthetic from B. anthracis
YWWLXPPX

SEQ ID NO: 22 - protein, 7 aa, Artificial
  Synthetic from B. anthracis
YWWLXPP